SEQUENCE LISTING (28 sequences)

SEQ ID NO: 1 - protein, 120 aa
  fragment type: internal
QVQLQESGPGLVRPSQTLSLTCTVSGFIFSDNYMYWVRQPPGRGLEWIGTISDGGSYTYYPDSVKGRVTMLRDTSKNQFSLRLSSVTAADTAVYYCARGYYRYEGAMDYWGQGSLVTVSS

SEQ ID NO: 2 - protein, 120 aa
  fragment type: internal
EVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSS

SEQ ID NO: 3 - protein, 120 aa
  fragment type: internal
EVQLVESGGGLVKPGGSLRLSCVASGFIFSDNYMYWVRQTPEKRLEWVATISDGGSYTYYPDSVKGRFTISRDNAKNNLYLQMSSLKSEDTAIYYCARGYYRYEGAMDYWGQGTSVTVSS

SEQ ID NO: 4 - protein, 112 aa
  fragment type: internal
DIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKVEIK

SEQ ID NO: 5 - DNA (cDNA), 31 bp; single-stranded, linear
ggaagcttagacagatgggggtgtcgttttg

SEQ ID NO: 6 - protein (cDNA), 34 aa
TGAGGAGACGGTGACCGTGGTCCCTTGGCCCCAG

SEQ ID NO: 7 - DNA (cDNA), 22 bp; single-stranded, linear
aggtsmarctgcagsagtcwgg

SEQ ID NO: 8 - DNA (cDNA), 34 bp; single-stranded, linear
tggaattcatggratggagctggrtcwtbhtctt

SEQ ID NO: 9 - DNA (cDNA), 33 bp; single-stranded, linear
tggaattcatgracttcdggytcaactkrrttt

SEQ ID NO: 10 - DNA (cDNA), 32 bp; single-stranded, linear
ggaagcttgaagatggatacagttggtgcagc

SEQ ID NO: 11 - DNA (cDNA), 24 bp; single-stranded, linear
gacattcagctgacccagtctcca

SEQ ID NO: 12 - DNA (cDNA), 32 bp; single-stranded, linear
ttgaattcggtgccagakcwsahatygtkatg

SEQ ID NO: 13 - DNA (cDNA), 32 bp; single-stranded, linear
ttgaattcggtggcagakcwsahatygtkctc

SEQ ID NO: 14 - DNA (cDNA), 32 bp; single-stranded, linear
ttgaattcggagctgatgggaacattgtaatg

SEQ ID NO: 15 - DNA (cDNA), 61 bp; single-stranded, linear
tgcctgtctcacccaatacatgtaattgtcactgaaatgaagccagacgmggagcggacag

SEQ ID NO: 16 - DNA (cDNA), 75 bp; single-stranded, linear
tgtaaatcttcccttcacactgtctggatagtaggtgtaactaccaccatcactaatggttgcaacccactcagg

SEQ ID NO: 17 - DNA (cDNA), 67 bp; single-stranded, linear
ggggtcccttggccccagtagtccatagccccctcgtacctatagtagcctcttgcacaaaaataga

SEQ ID NO: 18 - DNA (cDNA), 68 bp; single-stranded, linear
tggctgtctcacccaatacatgtaattgtcgctgaaaatgaagccagacacggtgcaggtcaggctca

SEQ ID NO: 19 - DNA (cDNA), 94 bp; single-stranded, linear
ttgctggtgtctctcagcattgtcactctccccttcacactgtctggatagtaggtgtaactaccaccatcactaatggttccaatccactcaa

SEQ ID NO: 20 - DNA (cDNA), 77 bp; single-stranded, linear
agacggtgaccaaggacccttggccccagtagtccatagccccctcgtacctatagtagcctcttgcacaataatag

SEQ ID NO: 21 - DNA (cDNA), 81 bp; single-stranded, linear
cttctgctggtaccaggccaagtagttcttctgatttgaactgtataaaacactttgactggacttacaggtgatggtcac

SEQ ID NO: 22 - DNA (cDNA), 45 bp; single-stranded, linear
gcttggcacaccagattccctagtggatgcccagtagatcagcag

SEQ ID NO: 23 - DNA (cDNA), 49 bp; single-stranded, linear
ccttggccgaacgtccacgaggagaggtattgatggcagtagtaggtgg

SEQ ID NO: 24 - DNA (cDNA), 17 bp; single-stranded, linear
gtaaaacgacggccagt

SEQ ID NO: 25 - DNA (cDNA), 16 bp; single-stranded, linear
aacagctatgaccatg

SEQ ID NO: 26 - DNA (cDNA), 24 bp; single-stranded, linear
tttacaatatcgagacaacagcaa

SEQ ID NO: 27 - DNA (cDNA), 24 bp; single-stranded, linear
ttgctgttgtctctcgattgtaaa

SEQ ID NO: 28 - protein, 112 aa
  fragment type: internal
NIVMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSWTFGGGTKLEIK

(19) United States
(12) Patent Application Publication
(10) Pub. No.: US 2003/0144483 A1
(43) Pub. Date: Jul. 31, 2003

(54) Humanized antibodies to Fc receptors for immunoglobulin G on human mononuclear phagocytes

(75) Inventors: Philip R. Tempest, Royston (GB); William J. Harris, Carnoustie (GB); Frank J. Carr, Balmedie (GB)

Correspondence Address: LAHIVE & COCKFIELD, 28 STATE STREET, BOSTON, MA, 02109, US

(73) Assignee: Medarex, Inc.

(21) Appl. No.: 10/229,335

(22) Filed: Aug. 26, 2002

Related U.S. Application Data
(63) Continuation of application No. 08/435,516, filed on May 4, 1995, now Pat. No. 6,500,931.

(51) Int. Cl. C07K 16/30

(52) U.S. Cl. 530/388.22

(57) ABSTRACT

Humanized antibodies are described which are specific to an Fc receptor (FcR). The humanized antibodies have at least a portion of a complementarity determining region (CDR) derived from a non-human antibody, e.g., murine, with the remaining portions being human in origin. The humanized antibodies can be used therapeutically as is or formulated as bifunctional molecules or immunotoxins.

### BACKGROUND

Human Fcγ receptors (FcγR) (reviewed in Fanger, M. W., et al. (1989) Immunology Today 10:92-99), of which there are three structurally and functionally distinct types (i.e., FcγRI, FcγRII and FcγRIII), are well-characterized cell surface glycoproteins that mediate phagocytosis or antibody-dependent cell cytotoxicity (ADCC) of immunoglobulin G (IgG) opsonized targets. Antibodies have been made which are directed towards FcγR for various purposes, e.g., targeting of immunotoxins to a particular target cell type, or radioimaging a particular target cell type. The antibodies typically have been murine antibodies.

Murine monoclonal antibodies are sometimes desirable for human therapeutic applications because the antibodies can be purified in large quantities and are free of contamination by human pathogens such as the hepatitis or human immunodeficiency virus. Murine monoclonal antibodies have been used in some human therapies, however, results have not always been desirable due to the development of an immune response to the “foreign” murine proteins. The immune response has been termed a human anti-mouse antibody or HAMA response (Schroff, R. et al. (1985), Cancer Res., 45, 879-885) and is a condition which causes serum sickness in humans and results in rapid clearance of the murine antibodies from an individual's circulation. The immune response in humans has been shown to be against both the variable and the constant regions of the murine immunoglobulin.

Recombinant DNA technology has provided the ability to alter antibodies by substituting specific immunoglobulin regions from one species with immunoglobulin regions from another species. Neuberger et al. (Patent Cooperation Treaty Patent Application No. PCT/GB85/00392) describes a process whereby the complementary heavy and light chain variable domains of an Ig molecule from one species may be combined with the complementary heavy and light chain Ig constant domains from another species. This process may be used to substitute the murine constant region domains to create a “chimeric” antibody which may be used for human therapy. A chimeric antibody produced as described by Neuberger et al. would have the advantage of having the human Fc region for efficient stimulation of antibody mediated effector functions, such as complement fixation, but would still have the potential to elicit an immune response in humans against the murine (“foreign”) variable regions.

Winter (British Patent Application Number GB2188538A) describes a process for altering antibodies by substituting the complementarity determining regions (CDRs) with those from another species. This process may be used to substitute the CDRs from the murine variable region domains of a monoclonal antibody with desirable binding properties (for instance to a human pathogen) into human heavy and light chain Ig variable region domains. These altered Ig variable regions may then be combined with human Ig constant regions to create antibodies which are totally human in composition except for the substituted murine CDRs. The “reshaped” or “humanized” antibodies described by Winter elicit a considerably reduced immune response in humans compared to chimeric antibodies because of the considerably less murine components. Further, the half life of the altered antibodies in circulation should approach that of natural human antibodies. However, as stated by Winter, merely replacing the CDRs with complementary CDRs from another antibody which is specific for an antigen such as a viral or bacterial protein, does not always result in an altered antibody which retains the desired binding capacity. In practice, some amino acids in the framework of the antibody variable region interact with the amino acid residues that make up the CDRs so that amino acid substitutions into the human Ig variable regions are likely to be required to restore antigen binding.

### SUMMARY OF THE INVENTION

The present invention pertains to humanized antibodies specific to an Fc receptor (FcR). The humanized antibodies have at least a portion of a complementarity determining region (CDR) derived from a non-human antibody, e.g., murine, with the remaining portions being human in origin. The use of humanized antibodies rather than murine antibodies in human therapy should alleviate some of the problems associated with the use of some murine monoclonal antibodies because only the substituted CDRs will be foreign to a human host's immune system.

The present invention further pertains to the use of humanized antibodies specific to an FcR as components in heteroantibodies, bifunctional antibodies, or immunotoxins. The humanized antibody specific to an FcR may be used in the same manner and for the same purpose as its corresponding murine counterpart. For example, the humanized anti-Fc receptor antibody of this invention can be used to treat cancer, allergies, and infectious and autoimmune diseases. Diagnostic applications of the antibodies include their use in assays for FcRI levels and assays for substances that influence FcR levels.

### BRIEF DESCRIPTION OF THE INVENTION

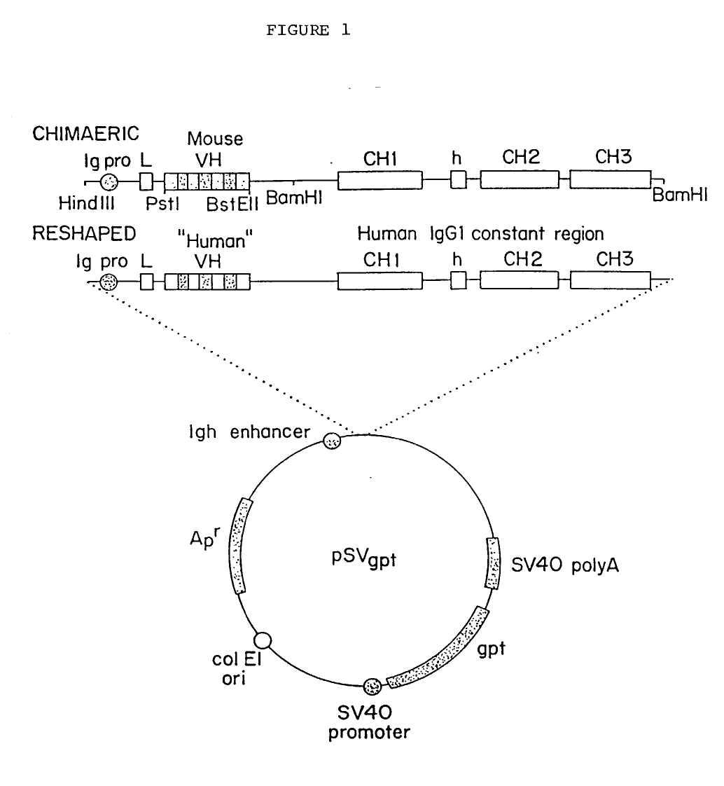

FIG. 1 depicts the vector used for expression of the humanized or chimeric 022 heavy chain gene.

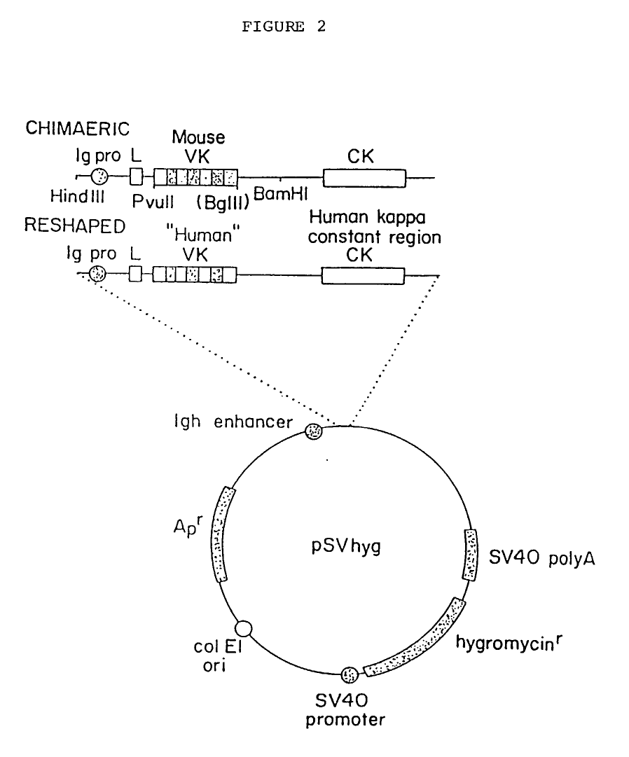

FIG. 2 depicts the vector used for expression of the humanized or chimeric 022 kappa chain gene.

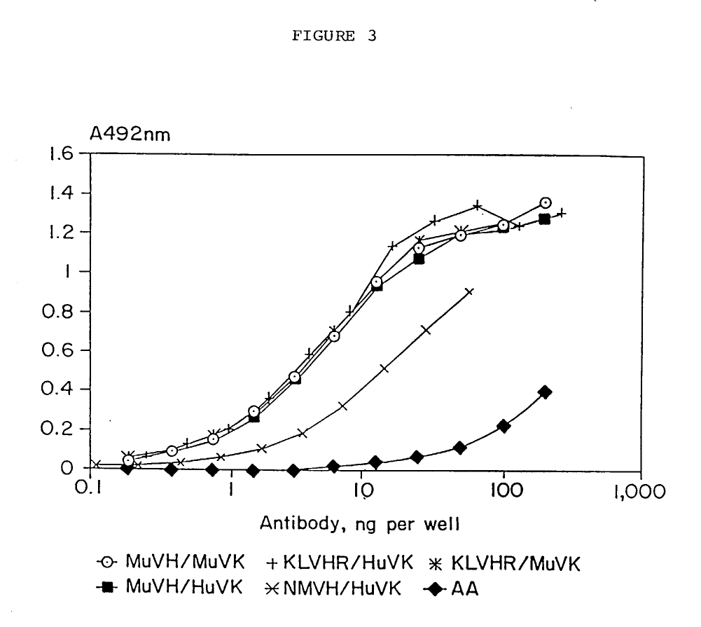

FIG. 3 depicts the binding of the test antibodies in the enzyme liked immunoassay described in the Example 1.

### DETAILED DESCRIPTION

The present invention pertains to a humanized antibody specific for an Fc receptor. The humanized antibody is made up of a human antibody having at least a portion of a complementarity determining region (CDR) derived from a non-human antibody. The portion is selected to provide specificity of the humanized antibody for a human Fc receptor. The humanized antibody has CDRs derived from a non-human antibody and the remaining portions of the antibody molecule are human.

The antibody may be a complete antibody molecule having full length heavy and light chains or any fragment thereof, e.g., Fab or (Fab′)₂ fragment. The antibody further may be a light chain or heavy chain dimer, or any minimal fragment thereof such as a Fv or a single chain construct as described in Ladner et al. (U.S. Pat. No. 4,946,778, issued Aug. 7, 1990), the contents of which is expressly incorporated by reference.

The human antibody of the present invention may be any human antibody capable of retaining non-human CDRs. The preferred human antibody is derived from known proteins NEWM and KOL for heavy chain variable regions (VHs) and REI for Ig kappa chain, variable regions (VKs). These proteins are described in detail in the examples below.

“Complementarity determining region” (CDR) is an art recognized term and the technique used for locating the CDRs within the described sequences also is conventional.

The portion of the non-human CDR inserted into the human antibody is selected to be sufficient for allowing binding of the humanized antibody to the Fc receptor. A sufficient portion may be selected by inserting a portion of the CDR into the human antibody and testing the binding capacity of the created humanized antibody using the enzyme linked immunosorbent assay (ELISA) described in the examples below.

All of the CDRs of a particular human antibody may be replaced with at least a portion of a non-human CDR or only some of the CDRs may be replaced with non-human CDRs. It is only necessary to replace the number of CDRs required for binding of the humanized antibody to the Fc receptor. The exemplified non-human CDR is derived from a murine antibody, particularly the CDR is derived from a monoclonal antibody (mab), mab 22. The mab 22 antibody is specific to the Fc receptor and further is described in U.S. patent application Ser. No. 07/151,450, filed on Feb. 2, 1988, and in Fanger et al. (U.S. Pat. No. 4,954,617, issued Sep. 4, 1988). The entire contents of the aforementioned pending application and issued patent are expressly incorporated by reference.

The CDRs are derived from a non-human antibody specific for a human Fc receptor. The CDRs can be derived from known Fc receptor antibodies such as those discussed in the Fanger et al. patent application and issued patent cited above (hereinafter Fanger et al.). The CDR may be derived from an antibody which binds to the Fc receptor at a site which is not blocked by human immunoglobulin G. The antibody also may be specific for the high affinity Fc receptor for human immunoglobulin G. Examples of antibodies from which the non-human CDRs may be derived are mab 32, mab 22, mab 44, mab 62, mab 197 and anti-FcRI antibody 62. The humanized mab 22 antibody producing cell line has been deposited at the American Type Culture Collection on Nov. 4, 1992 under the designation HA022CL1 and has the accession no. CRL 11177.

The present invention also pertains to humanized bifunctional molecules (i.e. molecules having two distinct binding specificities, at least one of which is humanized). For example, a bispecific molecule can have a binding specificity for a pathogen (e.g. virus, bacteria, fungi), pathogen infected cell, cancer (e.g. breast, ovarian, prostate, etc.) or other unwanted cell in a host and a binding specificity for an Fcγ receptor on an effector cell. A bispecific molecule may be comprised of two antibodies, in which event it is known as a “heteroantibody”. Procedures for generating bispecific molecules such as 520C9×H22, a humanized bispecific molecule against the HER 2/neu antigen of breast cancer cells is described in the attached Example 2. A humanized antigen binding region for an Fc receptor may be derived from a humanized anti-Fc receptor antibody as described above. Bifunctional molecules having an antibody portion specific for an Fc receptor are described in detail by Fanger et al.

It should be understood that the humanized antibodies of the present invention may be used in the same manner, e.g., as components of immunotoxins or heteroantibodies, as their corresponding non-humanized counterparts described by Fanger et al. The humanized antibodies further share the same utilities as their non-humanized counterparts. All aspects of the teachings of the Fanger et al. application and patent are incorporated by reference.

The humanized antibody of the present invention may be made by any method capable of replacing at least a portion of a CDR of a human antibody with a CDR derived from a non-human antibody. Winter describes a method which may be used to prepare the humanized antibodies of the present invention (UK Patent Application GB 2188638A, filed on Mar. 26, 1987), the contents of which is expressly incorporated by reference. The human CDRs may be replaced with non-human CDRs using oligonucleotide site-directed mutagenesis as described in the examples below.

The humanized antibody of the present invention may be made as described in the brief explanation below. A detailed method for production is set forth in the examples. It should be understood that one of ordinary skill in the art may be able to substitute known conventional techniques for those described below for the purpose of achieving the same result. The humanized antibodies of the present invention may be produced by the following process:

(a) constructing, by conventional techniques, an expression vector containing an operon with a DNA sequence encoding an antibody heavy chain in which the CDRs and such minimal portions of the variable domain framework region that are required to retain antibody binding specificity are derived from a non-human immunoglobulin, and the remaining parts of the antibody chain are derived from a human immunoglobulin, thereby producing the vector of the invention;

(b) constructing, by conventional techniques, an expression vector containing an operon with a DNA sequence encoding a complementary antibody light chain in which the CDRs and such minimal portions of the variable domain framework region that are required to retain donor antibody binding specificity are derived from a non-human immunoglobulin, and the remaining parts of the antibody chain are derived from a human immunoglobulin, thereby producing the vector of the invention;

(c) transfecting the expression vectors into a host cell by conventional techniques to produce the transfected host cell of the invention; and

(d) culturing the transfected cell by conventional techniques to produce the altered antibody of the invention.

The host cell may be cotransfected with the two vectors of the invention, the first vector containing an operon encoding a light chain derived polypeptide and the second vector containing an operon encoding a heavy chain derived polypeptide. The two vectors contain different selectable markers, but otherwise, apart from the antibody heavy and light chain coding sequences, are preferably identical, to ensure, as far as possible, equal expression of the heavy and light chain polypeptides. Alternatively, a single vector may be used, the vector including the sequences encoding both the light and the heavy chain polypeptides. The coding sequences for the light and heavy chains may comprise cDNA or genomic DNA or both.

The host cell used to express the altered antibody of the invention may be either a bacterial cell such as Escherichia coli, or a eukaryotic cell. In particular a mammalian cell of a well defined type for this purpose, such as a myeloma cell or a Chinese hamster ovary cell may be used.

The general methods by which the vectors of the invention may be constructed, transfection methods required to produce the host cell of the invention and culture methods required to produce the antibody of the invention from such host cells are all conventional techniques. Likewise, once produced, the humanized antibodies of the invention may be purified according to standard procedures of the art, including cross-flow filtration, ammonium sulphate precipitation, affinity column chromatography, gel electrophoresis and the like.

It should be understood that the humanized antibodies of this invention perform in a manner which is the same or similar to that of the non-humanized versions of the same antibodies. It also is noted that the humanized antibodies of this invention may be used for the design and synthesis of either peptide or non-peptide compounds (mimetics) which would be useful for the same therapy as the antibody (Saragobi et al., Science 253:792-795 (1991)), the contents of which is expressly incorporated by reference.

The following examples are provided as a further illustration of the present invention and should in no way be construed as being limiting.

### EXAMPLES

In the following examples all necessary restriction and modification enzymes, plasmids and other reagents and materials were obtained from commercial sources unless otherwise indicated.

In the following examples, unless otherwise indicated, all general recombinant DNA methodology was performed as described in “Molecular Cloning, A Laboratory Manual” (1982) Eds T. Maniatis et al., published by Cold Spring Harbor Laboratory Press, Cold Spring Harbor, N.Y., the contents of which is expressly incorporated by reference.

In the following examples the following abbreviations were employed:

| | |
|---|---|
| dCTP | deoxycytidine triphosphate |
| dATP | deoxyadenosine triphosphate |
| dGTP | deoxyguanosine triphosphate |
| dTTP | deoxythymidine triphosphate |
| DTT | dithiothreitol |
| C | cytosine |
| A | adenine |
| G | guanine |
| T | thymine |
| PBS | phosphate buffered saline |
| PBST | phosphate buffered saline |
| | containing 0.05% Tween 20 (pH 7.5) |

### Example 1

### Production of Humanized Antibodies Specific for an Fc Receptor

The source of the donor CDRs used to prepare the humanized antibody was a murine monoclonal antibody, mab 22, which is specific for the Fc receptor. A mab 22 hybridoma cell line (022WCL-1) was established. Cytoplasmic RNA was prepared from the mab 22 cell line using the method described by Favoloro et al. (Methods in Enzymology 65, 718-749 (1980)), the contents of which is expressly incorporated by reference. The cDNA was synthesized using IgGI and kappa constant region primers. The primer CG1FOR was used for the heavy chain variable (VH) region and the primer CK2FOR was used for the Ig kappa chain variable region (VK). The cDNA synthesis reactions mixtures consisted of 1 μg RNA, 0.5 μM CG1FOR or CK2FOR, 250 μM each of dATP, dCTP, dGTP, and dTTP, 50 mM Tris HCl (pH 7.5), 75 mM KCl, 10 mM dithiothreitol, 3 mM MgCl₂ and 20μ RNA guard (sold by Pharmacia, Milton Keynes, U.K.) in a total volume of 50 μl. The samples were heated at 72° C. for two minutes and slowly cooled to 37° C. Murine moloney leukemia virus reverse transcriptase (100 μl —sold by Life Technologies, Paisley, U.K.) was added to the samples and the transcriptase containing samples were incubated at 42° C. for sixty minutes.

VH and VK cDNAs were then amplified using the polymerase chain reaction (PCR) as described by Saiki et al. (Science 239, 487-491 (1988)), the contents of which is expressly incorporated by reference. The primers used in the above steps were as follows:

CG1FOR (SEQ ID NO:5) 5′ GGAAGCTTAGACAGATGGGGGTGTCGTTTTG 3′

VH1FOR (SEQ ID NO:6) 5′ TGAGGAGACGGTGACCGTGGTCCCTTGGCCCCAG 3′

VH1BACK (SEQ ID NO: 7) 5′ AGGTSMARCTGCAGSAGTCWGG 3′

SH1BACK (SEQ ID NO:8) 5′ TGGAATTCATGGRATGGAGCTGGRTCWTBHTCTT 3′

SH2BACK (SEQ ID NO:9) 5′ TGGAATTCATGRACTTCDGGYTCAACTKRRTTT 3′

CK2FOR (SEQ ID NO:10) 5′ GGAAGCTTGAAGATGGATACAGTTGGTGCAGC 3′

VK1BACK (SEQ ID NO:11) 5′ GACATTCAGCTGACCCAGTCTCCA 3′

VK5BACK (SEQ ID NO:12) 5′ TTGAATTCGGTGCCAGAKCWSAHATYGTKATG 3′

VK6BACK (SEQ ID NO:13) 5′ TTGAATTCGGTGGCAGAKCWSAHATYGTKCTC 3′

VK7BACK (SEQ ID NO:14) 5′ TTGAATTCGGAGCTGATGGGAACATTGTAATG 3′

Restriction sites incorporated in primers to facilitate cloning are underlined.

The PCR amplification of murine Ig DNA was conducted using the methodology described by Orlandi et al. (Proc. Natl. Acad. Sci USA 86, 3833-3838 (1989), the contents of which is expressly incorporated by reference. The DNA/primer mixtures consisted of RNA/cDNA hybrid (10 μl) and 25 pmol each of CG1FOR and SH1BACK or SH2BACK for PCR amplification of VH. The DNA/primer mixtures consisted of RNA/cDNA hybrid (10 μl) and 25 pmol each of CK2FOR and VK1BACK, VK5BACK, VK6BACK, VK7BACK for PCR amplification of VK. dATP, dCTP, dGTP and dTTP (250 μM each), 10 mM Tris HCl (pH 8.3), 60 mM KCl, 1.5 mM MgCl₂, 0.01% (w/v) gelatin, 0.01% (v/v) Tween 20, 0.01% (v/v) NP40 and 2.5μ Amplitaq (sold by Cetus, Beaconsfield, U.K.) were added to the samples in a final volume of 50 μl. The samples were subjected to 25-30 thermal cycles of PCR at 94° C. for thirty seconds, 55° C. for thirty seconds, 72° C. for one minute and a final cycle at 72° C. for five minutes.

The amplified VH and VK DNAs were run on a low melting point agarose gel and purified by Elutip-d column chromatography (sold by Schleicher and Schueell, Anderman, Walton, U.K.) for cloning and sequencing. The purified VH DNAs were cut with Eco I or Pst I and Hind III and cloned into M13mp18 and mp19 (sold by Pharmacia, Milton Keynes, U.K.). The purified VK DNAs were cut with Pvu II or Eco I and Hind III and cloned into M13mp18 and mp19. For general cloning methodologies see Sambrook et al. (Molecular Cloning: A Laboratory Manual, Cold Spring Harbor Laboratory, Cold Spring Harbor, N.Y. (1989)), the contents of which are expressly incorporated by reference. The resulting collection of clones were sequenced by the dideoxy method using T7 DNA polymerase (sold by Pharmacia, Milton Keynes, U.K.) as described by Sanger et al. (Proc. Natl. Acad. Sci. USA 74, 5463-5467, (1979)), the contents of which are expressly incorporated by reference.

From the sequences of the O22 VH and VR domains the CDR sequences were determined with reference to the database of Kabat et al. (“Sequences of Proteins of Immunological Interest” US Department of Health and Human Services, US Government Printing Office), the contents of which is expressly incorporated by reference, and utilizing computer assisted alignment with other VH and VK sequences.

Transfer of the murine O22 CDRs to human frameworks was achieved by oligonucleotide site-directed mutagenesis as described by Nakamye et al. (Nucleic Acids Res 14, 9679-9687 (1986)), the contents of which is expressly incorporated by reference. The primers used were as follows:

| | | |
|---|---|---|
| KLVHCDR1 | 5′TGCCTGTCTCACCCAATACATGTAA: | (SEQ ID NO:15) |
| | TTGTCACTGAAATGAAGCCAGACGMG | |
| | GAGCGGACAG | |
| KLVHCDR2 | 5′TGTAAATCTTCCCTTCACACTGTCT: | (SEQ ID NO:16) |
| | GGATAGTAGGTGTAACTACCACCATC | |
| | ACTAATGGTTGCAACCCACTCAGG | |
| KLVHCDR3 | 5′GGGGTCCCTTGGCCCCAGTAGTCCA: | (SEQ ID NO:17) |
| | TAGCCCCCTCGTACCTATAGTAGCCT | |
| | CTTGCACAAAAATAGA | |
| NMVHCDR1 | 5′TGGCTGTCTCACCCAATACATGTAA: | (SEQ ID NO:18) |
| | TTGTCGCTGAAAATGAAGCCAGACAC | |
| | GGTGCAGGTCAGGCTCA | |
| NMVHCDR2 | 5′TTGCTGGTGTCTCTCAGCATTGTCA: | (SEQ ID NO:19) |
| | CTCTCCCCTTCACACTGTCTGGATAG | |
| | TAGGTGTAACTACCACCATCACTAAT | |
| | GGTTCCAATCCACTCAA | |
| NMVHCDR3 | 5′AGACGGTGACCAAGGACCCTTGGCC: | (SEQ ID NO:20) |
| | CCAGTAGTCCATAGCCCCCTCGTACC | |
| | TATAGTAGCCTCTTGCACAATAATAG | |
| HuVKCDR1 | 5′CTTCTGCTGGTACCAGGCCAAGTAGT | (SEQ ID NO:21) |
| | TCTTCTGATTTGAACTGTATAAAACA | |
| | CTTTGACTGGACTTACAGGTGATGGT | |
| | CAC | |
| HuVKCDR2 | 5′GCTTGGCACACCAGATTCCCTAGTGG: | (SEQ ID NO:22) |
| | ATGCCCAGTAGATCAGCAG | |
| HuVKCDR3 | 5′CCTTGGCCGAACGTCCACGAGGAGAG: | (SEQ ID NO:23) |
| | GTATTGATGGCAGTAGTAGGTGG | |

The primer for NMVHCDR1 was extended to include a change of NEWM residues Ser 27 Thr 28 to Phe 27 Ile 28. The primer for NMVHCDR2 was extended to include a change of NEWM residue Val 71 to Arg 71.

The DNA templates used for mutagenesis of VHs comprised human framework regions from the crystallographically solved protein NEW described by Saul et al. (J. Biol. Chem. 53, 585-597 (1978)) or KOL described by Schmidt et al. (Z. Physical Chem. 364, 713-747 (1983)). The DNA templates used for mutagenesis of VKs comprised human framework regions from the crystallographically solved protein REI described by Epp et al. (Eur. J. Biochem. 45, 513-524 (1974)). The contents of each of the aforementioned references are expressly incorporated by reference.

M13 based templated M13VHPCR1 (for NEWMVH), M13VHPCR2 (for KOLVH) and M13VKPCR2 (for REIVK) comprising human frameworks with irrelevant CDRs were prepared as described by Riechmann et al. (Nature 332, 323-327 (1988)), the contents of which are expressly incorporated by reference. Oligonucleotide site-directed mutagenesis was carried out using the following protocol. A 5-fold molar excess of each phosphorylated mutagenic oligonucleotide was added along with the universal M13 sequencing primer (5′-GTAAAACGACGGCCAGT) (SEQ ID NO:24). All of the primers were annealed in 20 μl 0.1M TrisHCl (pH 8.0) and 10 mM MgCl₂ by heating to 70-85° C. for two minutes and slowly cooling to room temperature. 10 mM DTT, 1 mM ATP, 40 μM each of dATP, dCTP, dGTP and dTTP, 2.5μ T7 DNA polymerase (sold by United States Biochemicals) and 0.5μ T4DNA ligase (sold by Life Technologies, Paisley, U.K.) was added to the annealed DNA in a reaction volume of 30 μl and incubated at 22°-37° C. for one to two hours. The newly extended and ligated strand was preferentially amplified over the parental strand in a thermostable DNA polymerase directed reaction using the M13 reverse sequencing primer (5′ AACAGCTATGACCATG) (SEQ ID NO:25). The reverse sequencing primer is not complementary to the parental strand. The reaction mixture of 50 μl contained 1 μl extension/ligation product, 25 pmol M13 reverse sequencing primer, 250 μM each of dATP, dCTP, dGTP and dTTP, 1μ Vent DNA polymerase (sold by New England Biolabs, Bishop's Stortford, U.K.) or 2.5μ Amplitaq (sold by Cetus, Beaconsfield, U.K.) in the appropriate buffer supplied by the enzyme manufacturer and was subjected to thirty thermal cycles of 94° C., 30 s, 55° C., 30 s, 75° or 72° C., 90 s; ending with 5 min at 72° C. A 4 μl aliquot of this sample was then amplified by PCR using both M13 universal and reverse sequencing primers in a reaction mixture of 50 μl containing 25 pmol of each primer, 250 μM each of dATP, dCTP, dGTP and dTTP, 2.5μ Amplitaq (Cetus) in the buffer supplied by the enzyme manufacturer. Amplified DNAs were digested with HindIII and BamHI and cloned into M13mp19 and sequenced.

Mutagenesis of M13VHPCR2 KOL VH residue Leu71 to Arg71 was by the overlap/extension PCR method of Ho et al. (Gene, 77, 51-55 (1989)), the contents of which is expressly incorporated by reference. The overlapping oligonucleotides used were 5′-TTTACAATATCGAGACAACAGCAA (SEQ ID NO:26) and 5′-TTGCTGTTGTCTCTCGATTGTAAA (SEQ ID NO:27).

The amino acid sequences of the humanized antibodies were compared to the known murine antibodies as shown in FIGS. 1 and 2. The CDR replaced VH and VK genes were cloned into expression vectors pSVgpt and pSVhyg as shown in FIGS. 3 and 4 as described by Orlandi et al. (cited supra). The CDR replaced NEWMVH and KOLVH genes together with the Ig heavy chain promoter, appropriate splice sites and signal peptide sequences were excised from M13 by digestion with HindIII and BamHI and cloned into the pSVgpt expression vector containing the murine Ig heavy chain enchancer, the gpt gene for selection in mammalian cells and genes for replication and selection in E. coli. The plasmid also contains a human IgGI constant region as described by Takahashi et al. (Cell 29, 671-675 (1982)). The construction of the kappa chain expression vector was essentially the same except that the gpt gene was replaced by the hygromycin resistance gene and contains a human kappa constant region (Hieter et al., Cell 22, 197-207 (1980)). The contents of each of the aforementioned references are expressly incorporated by reference.

Approximately 5 μg of each heavy chain expression vector and 10 μg of the kappa chain expression vector were digested with PvuI. The DNAs were mixed together, ethanol precipitated and dissolved in 25 μl water. Approximately 5-10×10⁶ NSO cells (from European Collection of Animal Cell Cultures, Porton Down, U.K.) were grown to semi-confluency in Dulbecco's modified Eagle's medium (DMEM) plus 10% fetal calf serum (Myoclone plus, Gibco, Paisley, Scotland), harvested by centrifugation and resuspended in 0.5 ml DMEM together with the digested DNA in a cuvette. After five minutes in ice, the cells were given a single pulse of 170V at 960 μF (Gene-Pulser, Bio-Rad, Richmond, Calif.) and left in ice for a further twenty minutes. The cells were then put into 20 ml DMEM+supplemented with 10% FCS and allowed to recover for twenty-four to forty-eight hours. After this time, the cells were distributed into a 24-well plate and selective medium was applied (DMEM, 10% FCS, 0.8 μg/ml mycophenolic acid and 250 μg/ml xanthine). After three to four days, the medium and dead cells were removed and replaced with fresh selective medium. Transfected clones were visible with the naked eye ten days later.

The presence of human antibody in the medium of wells containing gpt+ transfectants was measured using conventional enzyme linked immunosorbent assay (ELISA) techniques. Wells of a microtitre plate (Immolon, Dynatech, Chantilly, Va.) were coated with 100 ng goat anti-human IgG antibodies (SeraLab, Crawley Down, U.K.) in 100 μl 50 mM carbonate buffer pH 9.6. After washing with PBST (Phosphate buffered saline pH 7.2 containing 0.05% Tween 20) culture medium in 100 μl PBST (5-50 μl) was added to each well for one hour at 37° C. The wells were then emptied, washed with PBST and 100 μl of 1:1000 dilution peroxidase conjugated goat anti-human kappa constant region antibodies (SeraLab, Crawley Down, U.K.) were added for one hour at 37° C. The wells were emptied, washed with PBST and 100 μl OPD substrate buffer (400 μg/ml Q-phenylenediamine in 24 mM citrate/42 mM sodium phosphate pH 5. and 0.0003% (v/v) H₂O₂) was added. The reaction was stopped after a few minutes by the addition of 12.5% H₂SO₄ (25 μl) and the absorbance at 492 nm was measured.

The antibody secreting cells were expanded and antibody was purified from the culture medium by protein A affinity chromatography as described by Harlow and Lane (Antibodies: A Laboratory Manual, Cold Spring Harbor Laboratory, Cold Spring Harbor, N.Y.), the contents of which is expressly incorporated by reference.

The binding of the antibodies to antigen was measured by ELISA. Wells of a microtitre plate (Immunlon 1, Dynatech, Chantilly, Va.) were coated with 200 ng goat anti-human IgM antibodies (Sera-lab, Crawley Down, U.K.) in 100 μl 50 mM carbonate buffer pH 9.6 at 37° C. for at least one hour. Wells were emptied and washed once with PBST and blocked with 1% BSA in PBS at room temperature for thirty minutes. The wells were emptied and washed with PBST and Cos supernatant containing FcRI/IgM fusion protein was added and incubated for one hour at room temperature. Wells were then emptied and washed three times with PBST and test antibodies diluted in 1% BSA/PBS were added and incubated for one hour at room temperature. In addition, each well contained 2 μg human IgGI, lambda antibody (Sigma, Poole, U.K.) The wells were then emptied, washed three times with PBST and 40 ng peroxidase goat anti-human kappa constant region antibodies (Sera-Lab, Crawley Down, U.K.) in 100 μl 1% BSA/PBS added to each well. After incubation for one hour at room temperature, the wells were emptied, washed three time with PBST and 10 μl HPD substrate buffer was added. The reaction was stopped by the addition of 25 μl of 12.5% H₂SO₄ to each well. The absorbance at 492 nm was measured and is depicted in FIG. 5. The test antibodies were the antibody containing irrelevant CDRs (AA), the fully humanized KOL/REI based antibody (KLVHR/HuVK), the mix and match derivatives of the humanized antibody (KLVHR/MuVK and MuVH/HuVK), the humanized NEWM/REI based antibody (NMVK/HuVK) and the chimeric antibody (MuVH/MuVK).

### Example 2

### Production of Bispecific Antibody Comprising Antibodies Specific for an Fc Receptor and an Anti-her 2 neu Antibody

Monoclonal Antibodies

The anti-FcγRI mAbs, M22, M32.2 and 197 were purified from hybridoma supernatant by ion exchange chromatography and DZ33, a human anti-HIV-1 IgG1 mAb, was purified from hybridoma supernatant by protein A affinity chromatography (Pharmacia, Piscataway, N.J.) and gel filtration. M32.2 was deposited at the American Type Culture Collection, 12301 Parklawn Drive, Rockville, Md. 20852, on Jul. 1, 1987 and has been designated with ATCC Accession No. HB9469.

Cell Lines

The murine mycloma NSO (ECACC 85110503) is a non-Ig synthesizing line and was used for the expression of recombinant mAbs. NSO cells were cultivated in DMEM plus 10% fetal bovine serum (FBS, Gibco, Paisley, U.K.). SKBR-3 is a human breast carcinoma cell line which overexpresses the HER2/neu protooncogene (ATCC, Rockville, Md.) and was cultivated in Iscove's Modified Dulbecco's Medium (IMDM, Gibco, Grand Island, N.Y.). U937 is a monocytoid cell line that expresses FcγRI and was obtained from ATCC and grown in RPM-1640 plus 10% FBS (Gibco, Grand Island, N.Y.).

Cloning Murine Immunoglobulin V Region Genes

Cytoplasmic RNA from the murine hybridoma 22 was prepared as described in Favaloro et al. (Favaloro, J., R. Treisman and R. Kamen (1982) Transcription maps of polyoma-specific RNA: analysis by two-dimensional S1 gel mapping. Meth. Enzymol. 65:718). The Ig V region cDNAs were made form RNA via reverse transcription initiated from primers CG1FOR, (SEQ ID NO:5) 5′-GGAAGCTTAGACAGATGGGGGTGTCGTTTTG, (encoding amino acids 115-122 of the murine IgG1 CH1 domain and a Hind III site) and CK2FOR, (SEQ ID NO:10) 5′-GGAAGCTTGAAGATGGATACAGTTGGTGCAGC, (encoding amino acids 1-118 of the murine kappa constant domain and a Hind III site). The cDNA synthesis was performed under standard conditions using 100 U MMLV reverse transcriptase (Life Technologies, Paisley, UK). The V_{H} and V_{κ} cDNAs were amplified by PCR, (Orlandi, R., D. H. Güssow, P. T. Jones and G. Winter (1989) (Cloning immunoglobulin variable domains for expression by the polymerase chain reaction), Proc. Natl. Acad. USA 86:3833), using the cDNA primers in concert with SH2BACK, (SEQ. ID. NO:9) 5′-TGGAATTCATGRACTTCDGGYTCAACTKRRTTT (encoding a consensus sequence of amino acids −20 to −12 of some V_{H} signal peptides and an EcoR I site) and VK7BACK, (SEQ. ID. NO:14) 5′-TTGAATTCGGAGCTGATGGGAACATTGTAATG (encoding amino acids −4 to −1 of the signal peptide and residues 1-4 of some murine V_{κ} domains and an EcoR I site). Amplified V_{H} and V_{κ} DNA were purified, cloned into M13, and sequenced by the dideoxy method using T7 DNA polymerase (Pharmacia, Piscataway, N.J.).

Construction of Chimeric Antibody Genes

To facilitate cloning of murine V region DNA into expression vectors, restriction sites were placed in close proximity to the termini of both M22 V region genes. For V_{H}, a 5′ PstI site and a 3′ BstEII site were introduced into a cloned murine V_{H} gene by PCR using VH1BACK and VH1FOR (Id.). For V_{κ} a 5′ PvuII site and a 3′ Bgl II site were introduced into a cloned murine V_{κ} gene by PCR using primers VK1BACK and VK1FOR (Id.). In some instances, these primers changed one or more amino acids from those naturally occurring (see FIG. 1). These V region genes (ChVH and ChVK) were cut with the appropriate restriction enzymes and cloned into M13VHPCR1 and M13VKPCR1 (Id.) which contain an Ig promoter, signal sequence and splice sites. The DNA were excised from M13 as HindIII-BamHI fragments and cloned into the expression vectors pSVgpt and pSVhyg containing human IgG1, (Takahashi, N. et al., (1982), Structure of human immunoglobulin gamma genes: implications for evolution of a gene family, Cell, 29:671), and human kappa constant, (Hieter, R. A. et al., (1980) Cloned human and mouse kappa immunoglobulin constant and J region genes conserve homology in functional segments, Cell 22:197), region genomic DNA.

Construction of Humanized Antibody Genes

Two humanized heavy chains were constructed and were based on human V_{H}s of NEWM, (Poljak, R. J. et al., Amino acid sequence of the V_{H} region of a human mycloma immunoglobulin, (IgG New), Biochemistry, 16:3412), and KOL,(Marquat, M. et al., (1980) Crystallographic refinement and atomic models of the intact immunoglobulin molecule Kol and its antigen-binding fragment at 3.0A and 1.9A resolution, J. Mol. Biol. 141:369. The humanized light chain was derived from the human Bence-Jones protein REI, (Epp, O. et al, (1974) Crystal and molecular structure of a dimer composed of the vandible portion of the Bence-Jones protein REI, Eur. J. Biochem. 45:513), with some framework region (FR) changes. The modifications were made to make the V_{κ} domain more typical of human subgroup I, and included replacement of Thr39, Leu104, Gln105 and Thr107 with Lys39, Val104, Glu105 and Lys107. In addition, Met4 was changed to Leu4 to accommodate a PvuII restriction site.

DNA containing the NEWM V_{H} and REI V_{κ} FRs with irrelevant CDRs were cloned into the vectors M13VHPCR1 and M13VKPCR1 (Favaloro et al. Supra). DNA encoding the KOL V_{H} was constructed by a series of sequential PCRs, using oligodeoxyribonucleotides encoding KOL FR amino acids and irrelevant CDRs. The constructs were then cloned into M13VHPCR1.

Oligodeoxyribonucleotides were synthesized to encode the mAB M22 CDRs which were flanked by nucleotides corresponding to the human FRs. For the humanized V_{H} based on NEWM, the primers included murine FR amino acids Phe27, IIe28 and Arg71 since these were likely to influence antigen binding, (Chothia, C. and A. M. Lesk (1987), Canonical structures for the hypervariable regions of immunoglobulins, J. Mol. Biol., 196:901; Tramontano, A. et al., (1990), Framework residue 71 is a major determinant of the position and conformation of the second hypervariable region in V_{H} domains of immunoglobulins, J. Mol. Biol., 215:175). For the humanized V_{κ}, murine amino acid Phe71 was similarly included as a residue capable of affecting affinity, (Foote, J. and G. Winter, (1992), Antibody framework residues affecting the conformation of the hypervariable loops, J. Mol. Biol. 224:487. No murine FR residues were included in the KOL V_{H}. Oligodeoxyribonucleotides were 5′-phosphorylated and with the M13 universal forward primer annealed to the human V region genes cloned in M13 in reactions containing M13 ssDNA template. The DNA was extended and ligated with 2.5 U T7 DNA polymerase (United States Biochemicals, Cleveland, Ohio) and 0.5 U T4 DNA ligase (Gibco BRL, Grand Island, N.Y.). The mutated strand was preferentially amplified from the extension/ligation mixture using M13 reverse sequencing primer with 1 U Vent DNA polymerase (New England Biolabs, Beverly, Mass.) and was then amplified by PCR using both M13 forward and reverse primers. Product DNA was cut with BamH1 and HindIII, cloned into M13 and triple CDR-grafted mutants identified by DNA sequencing.

M13 clones containing the humanized V regions were sequenced in their entirety to ensure the absence of spurious mutations. RF DNA from the confirmed clones was digested with HindIII and BamHI, cloned into pSVgpt or pSVhyg and human IgG1 or human kappa constant regions added exactly as described for the construction of the chimeric antibody genes.

Expression and Purification of Recombinant mAbs

Heavy (5 μg) and light (10 μg) chain expression vectors were digested with PvuI, ethanol precipitated and dissolved in 50 μl water. NSO cells (1-2×10⁷) were harvested by centrifugation, resuspended in 0.5 ml DMEM and mixed with the DNA in a 0.4 cm electroporation cuvette. After 5 min. on ice the cells were given a single pulse of 170 V, 960 μF (GenePulser, Bio-Rad, Melville, N.Y.) and incubated further for 15 min. on ice. The cells were allowed to recover in DMEM for 24-48 hours. The medium was then made selective by the addition of mycophenolic acid (0.8 μg/ml) and xanthine (250 μg/ml). Aliquots of 200 μl were distributed into 96-well plates. After a further 10-12 days, cells from the wells containing the highest levels of antibody measured by ELISA were selected and cloned by limiting dilution.

Antibodies were purified from overgrown cultures by protein A affinity chromatography (Boehringer Mannheim, Lewes, U.K.) Concentrations were determined by measuring A_{280 nm} and confirmed by ELISA and SDS-PAGE.

ELISA for Measurement of Antibody Binding

The wells of a microtiter plate were coated with goat anti-human IgM antibodies (Sera-Lab, Crawley Down, U.K.) in 50 mM bicarbonate buffer, pH 9.6. The plate was blocked with 1% BSA and followed by the addition of a soluble fusion protein consisting of the extracellular domain of human FcγRI and human IgM heavy chain (sFcγRI-μ) obtained from transiently transfected COS cells (the expression vector was kindly provided by Dr. Brian Seed, Massachusetts General Hospital, Boston, Mass.). Recombinant 22 or control mAbs were then added in the presence of excess (2.2 μg/well) human IgG1 antibodies (Sigma, St. Louis, Mo.) that contained λ light chains to block the non-specific binding of the test mAbs via their Fc portion. Bound 22 mAbs were detected with peroxidase-labeled goat anti-human kappa chain antibodies (Sera-Lab, Crawley Down, U.K.) and o-phenylenediamine.

Fluoresceination of Antibodies

The pH of mAb solution was adjusted to 9.3 by the addition of 0.1M Na₂CO₃. Fluorescein iso-thiocyanate (FITC) (Sigma, St. Louis, Mo.) was dissolved in DMSO at a concentration of 2 mg/ml. Forty μg of FITC was added for each milligram of mAb and incubated for two hours at room temperature. The fluoresceinated mAb was separated from the free FITC by G-25 chromatography.

Preparation of Blood Cells

Buffy coats were prepared from heparinized whole venous blood. Whole blood was diluted with RPMI containing 5% dextran at a ratio of 2.5:1 (v/v). The erythrocytes were allowed to sediment for 45 minutes on ice, then the cells in the supernatant were transferred to a new tube and pelleted by centrifugation. The residual erythrocytes were removed by hypotonic lysis. The remaining lymphocytes, monocytes and neutrophils were kept on ice until use in binding assays. For some experiments, neutrophils were separated from mononuclear cells by ficoll hypaque (Pharmacia, Piscataway, N.J.) gradient separation. To up-regulate FcγRI, neutrophils and mononuclear cells were treated with cytokines. Cultures of mononuclear cells were incubated at 37° C., 5% CO₂ for 48 hours in teflon dishes at 4×10⁶ cells/ml of RPMI containing 2.5% normal human serum type AB (Sigma, St. Louis, Mo.) and 500 IRU/ml IFN-γ (R&D Systems, Minneapolis, Minn.). Neutrophils were cultured for 48 hours (37° C., 5% CO₂) in AIM V media (Gibco, Grand Island, N.Y.) with 50 ng/ml G-CSF (Kindly provided by R. Repp, U. of Erlanger, Germany) and 500 IRU/ml IFN-γ.

Flow Cytometry

Cell binding assays were performed using 96-well microtiter plates as previously described, (Guyre, P. M. et al., Monoclonal antibodies that bind to distinct epitopes on FcγR are able to trigger receptor function. J. Immunol., 143:1650). Briefly, cells were washed in PBS, pH 7.4 containing 2 mg/ml BSA and 0.05% NaN₃ (PBA), and adjusted to 2.0×10⁷ cells/ml with PBA. FITC-labeled and unconjugated antibodies were prepared in PBA. Cells (25 μl), antibody (25 μl) and human serum (25 μl), or human IgG (10 mg/ml, Sigma, St. Louis, Mo.) (25 μl), or PBA (25 μl) were added to the microtiter plate, and left on ice for 45-60 minutes. Unbound antibody was removed from the wells by washing the cells 3 times with PBA. The cells were fixed with 1% paraformaldehyde. Cell associated fluorescence was analyzed on a Becton Dickinson FACScan.

BsAb Coupling Procedure

BsAb were constructed using the method of Glennie et al, (Glennie, M. J. et al., (1987), Preparation and performance of bispecific F(ab′ gamma)², antibody containing thioether-linked Fab′ gamma fragments, J. Immunol., 139:2367. mAbs 22 (both murine and humanized) and 520C9 (anti-HER2/neu) antibodies were produced by in vitro cultivation of the respective hybridoma cells. The antibodies were separately digested with pepsin to F(ab′)₂, and subsequently reduced to Fab′ by addition of 10 mM mercaptoethanolamine (MEA) for 30 minutes at 30° C. The Fab′ fragments were applied to a Sephadex G-25 column equilibrated in 50 mM Na Acetate, 0.5 mM EDTA, pH 5.3 (4° C.). Ortho-phenylenedimaleimide (o-PDM, 12 mM) dissolved in dimethyl formamide and chilled in a methanol/ice bath was added (one half volume) to the murine 22 Fab′ in the case of M 22×520C9, and to 520C9 Fab′ in the case of H 22×520C9 and incubated for 30 minutes on ice. The Fab′-maleimide was then separated from free o-PDM on Sephadex G-25 equilibrated in 50 mM Na Acetate, 0.5 mM EDTA, pH 5.3 (4° C.). For preparation of the BsAbs, the M22 Fab′-maleimide was added to the 520C9 Fab′ or the 520C9 Fab′-maleimide was added to H22 Fab′ at a 1:1 molar ratio. The reactants were concentrated under nitrogen to the starting volume using a Diaflo membrane in an Amicon chamber (all at 4° C.). After 18 hours the pH was adjusted to 8.0 with 1M Tris-HCl, pH 8.0. The mixture was then reduced with 10 mM MEA (30 minutes, 30° C.) and alkylated with 25 mM iodoacetamide. The bispecific F(ab′)₂ was separated from unreacted Fab's and other products by a Superdex 200 (Pharmacia, Piscataway, N.J.) column equilibrated in PBS.

Antibody Dependent Cellular Cytotoxicity (ADCC)

The HER2/neu over-expressing human breast carcinoma cells, SKBR-3, were used as targets for lysis by cytokine activated neutrophils (see preparation of blood cells). Targets were labeled with 100 μCi of ⁵¹Cr for 1 hour prior to combining with neutrophils and antibodies in a U-bottom microtiter plate. After incubation for 5 hours at 37° C. supernatants were collected and analyzed for radioactivity. Cytotoxicity was calculated by the formula: % lysis=(experimental CPM−target leak CPM/detergent lysis CPM−target leak CPM)×100%. Specific lysis=% lysis with antibody−% lysis without antibody. Assays were performed in triplicate.

Superoxide Induction

U937 cells were used for measuring the ability of H22 to trigger a superoxide burst via FcγRI, (Pfefferkorn, L. C. and G. R. Yeaman (1994), Association of IgA-Fc receptors (Fc×R) with Fcε RIγ 2 subunits in U937 cells, J. Immunol. 153:3228; Hallet, H. B. and A. K. Campbell (1983). Two distinct mechanisms for stimulating of oxygen-radical production in polymorphonuclear leucocytes, Biochem J. 216:459). U937 cells were cultured for five days in RPMI-1640 (Gibco, Grand Island, N.Y.) with 10% FBS (Hyclone, Logan, Utah) in the presence of 100 U/ml IFN-γ (Genentech, S. San Francisco, Calif.) to induce differentiation and increased expression of FcγRI. On the day of the experiment, these differentiated cells were incubated for 20 minutes in fresh RPMI-1640 with 10% FBS at 37° C. The cells were then pelleted and resuspended at a concentration of 3×10⁶ cells/ml in PBS supplemented with 1 mM CaCl₂, 1 mM MgCl₂, 11 mM glucose, and 100 μg/ml BSA (Sigma, St. Louis, Mo.). To trigger the release of superoxide, 100 μl of cells were added to 100 μl of a reaction solution containing 0.1 mM luminol (Sigma, St. Louis, Mo.), 0.5 mM sodium vanadate (Sigma, St. Louis, Mo.), and either mAb M22, H22, or 197 and placed in the luminometer at 22° C. Measurements of the spontaneous production of superoxide were made every 30 to 40 seconds starting immediately following the addition of the cells to the reaction solution in the luminometer. To compare the superoxide triggered by crosslinking FcγRI with M22, H22 or 197, each mAb was used at a concentration of 10 μg/ml. The production of superoxide in mV/sec was monitored for 20 minutes. MAb M22, M32.2 and 197 were added at various concentrations to establish the dose-responsiveness of superoxide production.

Results

Murine Ig V Region Genes

Ig V region cDNAs were prepared from M22 hybridoma RNA using primers specific for murine heavy and kappa constant regions and were amplified by PCR with the additional use of a series of primers based on sequences of known signal and/or 5′ sequences of mature V regions. PCR products of the expected sizes for V_{H} and V_{κ} were obtained using the SH2BACK/CG1FOR and VK7BACK/CK2FOR primer combinations. Amplified DNA was digested with appropriate restriction enzymes, cloned into M13 and the sequence in both directions determined from at least 24 independent clones. The deduced amino acid sequences are shown in SEQ. ID Nos. 29 and 30. The 4 N-terminal residues of V_{κ} are encoded by the VKBACK primer.

The M22 V_{H} and V_{κ} are members of murine heavy chain subgroup IIID and kappa subgroup I, (Kabat, E. A. et al., (1991), Sequences of Proteins of Immunological Interest, 5th Ed., U.S. Department of Health and Human Services), respectively. Apart from the residue at L97, the amino acid sequence of the M22 V_{κ} is identical to that from the murine anti-IgG mAb A17 (Shlomchik, M. et al., Variable region sequences of murine IgM anti-IgG monoclonal autoantibodies (rheumatoid factors). II Comparison of hybridonias derived by lipopolysaccharide stimulation and secondary protein immunization, J. Exp. Med. 165:970).

Humanized mAbs and Initial Characterization of Their Binding

M22 V_{H} FR showed greater homology (79%) to KOL (human subgroup III) than to NEWM (57%) (human subgroup II). To see how this difference might affect binding, heavy chains were constructed based either on NEWM V_{H} including the murine residues Phe27, Ile28 and Arg71, or on KOL V_{H} with no murine FR amino acids. Both humanized V_{H} were partnered with the same REI-derived humanized light chain.

The affinity of the humanized mAbs was initially assessed by ELISA measuring the binding to FcγRI/IgM heavy chain fusion protein. The data showed that the KOL V_{H}/REI V_{κ} mAb had the same binding as the chimeric mAb whereas the NEWM V_{H}/REI V_{κ} mAb exhibited an approximate 5-fold lower affinity. The low binding of a nonspecific human IgG1 mAb showed that >95% of binding of the humanized mAbs was via the Fv portion rather than through the Fc domain.

While additional changes to the NEWM FR would be expected to restore binding affinity these could create novel epitopes which might provoke an unwanted immunological response. We therefore chose the KOL V_{H}/REI V_{κ} mAb, designated H22, for a further examination of its binding characteristics.

Functional Characterization of mAbH22

A series of binding experiments were performed to establish the specificity and isotype of the H22 antibody. Peripheral blood leukocytes stained with fluorescein-conjugated M22 or H22 demonstrated specific binding to monocytes with approximately 10⁴ binding sites per cell. In contrast, lymphocytes or unstimulated neutrophils had little or no specific binding (Table 1):

TABLE 1
Specific Binding of H22 to Monocytes

| Antibody | Monocytes | Lymphocytes | PMNs |
|---|---|---|---|
| M22 | 10,000^{a} | <1000 | <1000 |
| H22 | 10,500 | <1000 | <1000 |

^{a}Antibody sites per cell, average of duplicates

To demonstrate that the H22 binds to FcγRI at the same site as M22 and that it also binds as a ligand at the Fc binding domain, competition experiments with two anti-FcγRI murine mAb (M22 and M32.2) and a human IgG1 mAb were performed. Unconjugated H22 and M22 competed equivalently for either the binding of fluorescinated M22 or fluoresceinated H22 in the presence of excess human IgG which saturated the Fc binding sites on FcγRI. As expected, the anti-FcγRI antibody M32.2 which binds to a different site on FcγRI than M22 (Guyre, P. M. et al., J. Immunol. 143:1650) was also unable to compete with the M22-FITC. In addition, the inhibition of H22-FITC by H22 and not by an irrelevant human IgG1 mAb confirmed the specificity of FcγRI binding via the V regions of H22.

H22, but not M22, was able to compete for Fc mediated binding to FcγRI by a fluorosceinated human IgG1. This experiment demonstrated that the Fc portion of H22 but not M22 bound to the Fc binding domain of FcγRI. This is consistent with the ability of the Fc portion of human IgG1 antibodies, but not murine IgG1, to bind FcγRI with high affinity.

Since the humanization of M22 was primarily to increase its immunotherapeutic potential, the binding activity of H22 to monocytes and cytokine-activated neutrophlils was determined in the presence of human serum. H22-FITC bound with similar affinity to FcγRI on monocytes in the presence or absence of human serum. In contrast, the Fc-mediated binding of an irrelevant human IgG-FITC was completely inhibited by human serum. Likewise, H22-FITC bound with similar affinity to IFN-γ-treated neutrophils in the absence and in the presence of human serum. Collectively, the data demonstrated that H22 binds both via its V regions to a site distinct from the Fc binding domain and via its Fc region to the ligand binding domain of FcγRI. The former binding activity effectively overcomes antibody blockade of human IgG1.

Functional Activity of H22 BsAb

The foremost application of anti-FcγRI antibodies for immunotherapy is the development of BsAbs which link FcγRI-bearing effector cells to a tumor cell, a virus, or a virally-infected cell. Such BsAb have been developed with M22; therefore, a comparison was made of the ability of the M22 anti-tumor BsAb (520C9×M22) and a corresponding H22 BsAb (520C9×H22) to mediate cytotoxicity. These BsAbs consisted of H22 or M22 Fab′ chemically conjugated to the Fab′ of an anti-HER2/neu antibody (520C9), and thus were specific for the effector cell trigger molecule FcγRI and the tumor antigen.

Comparison of M22-derived and H22-derived BsAbs was done by ADCC assays. M22- and H22-derived BsAbs mediated the killing of HER2/neu overexpressing SKBR-3 cells. Both the murine and humanized BsAbs exhibited similar levels of lysis of antigen bearing target cells. In addition, both BsAb retained ADCC activity in the presence of human serum, while excess M22 F(ab′)₂ resulted in complete inhibition of killing. Taken together these results show that the H22 BsAb-induced lysis is mediated through the M22 epitope and that the ADCC is FcγRI specific.

Finally, the ability of H22 and M22 to stimulate superoxide production by the monocyte-like cell line U937 was evaluated. M22, which binds to the FcγRI only by its V regions, induced a very low level oxygen burst, presumably because it is unable to cross-link the receptor efficiently. However, H22, which can cross-link FcγRI by binding as a ligand via its Fc domain and, additionally, as an antibody via its Fv, induced a more substantial release of superoxide.

Equivalents Those skilled in the art will recognize, or be able to ascertain using no more than routine experimentation, many equivalents to the specific embodiments of the invention described herein. Such equivalents are intended to be encompassed by the following claims.

1. A humanized antibody specific to an Fc receptor comprising:
a human antibody having at least a portion of a complementarity determining region derived from a non-human antibody, the portion being selected to provide specificity of the humanized antibody for a human Fc receptor.

2. The antibody of claim 1 wherein the portion is selected to provide specificity to a human Fc receptor such that the humanized antibody formed binds to the Fc receptor at a site which is not blocked by human immunoglobulin G.

3. The antibody of claim 1 wherein the portion is selected to provide specificity of the humanized antibody for the high affinity Fc receptor for human immunoglobulin G.

4. The antibody of claim 1 wherein the portion of a complementarity determining region is derived from a murine antibody.

5. The antibody of claim 1 wherein the portion of a complementarity determining region is derived from a monoclonal antibody selected from the group consisting of mab 32, mab 22, mab 44, mab 62, mab 197, and anti-FcRI antibody 62.

6. The antibody of claim 5 wherein the monclonal antibody is mab 22.

7. The antibody of claim 1 wherein at least a portion of all of the complementarity determining regions of the human antibody are derived from a non-human antibody.

8. The antibody of claim 7 wherein the entire portion of all of the complementarity determining regions of the human antibody are derived from a non-human antibody.

9. The antibody of claim 7 wherein the non-human antibody is mab 22.

10. The antibody of claim 8 wherein the non-human antibody is mab 22.

11. The antibody of claim 1 wherein the human antibody is derived from proteins selected from the group consisting of NEW, KOL, REI, and combinations thereof.

12. A bispecific molecule, comprising:
at least one humanized antigen binding region derived from a humanized anti-Fc receptor antibody, and
at least one antigen binding region specific for a target epitope.

13. The bispecific molecule of claim 12 wherein the humanized antigen binding region is derived from a humanized anti-Fc receptor antibody selected such that the binding of the humanized antibody to the human Fc receptor is not blocked by human immunoglobulin G.

14. The bispecific molecule of claim 13 wherein the humanized anti-Fc receptor antibody is specific for the high affinity Fc receptor for human immunoglobulin.

15. The bispecific molecule of claim 12 wherein the humanized antigen binding region has at least a portion of a complementarity determining region derived from a non-human antibody.

16. The bispecific molecule of claim 15 wherein the portion of a complementarity determining region is derived from a murine antibody.

17. The bispecific molecule of claim 15 wherein the portion of a complementarity determining region is derived from a monoclonal antibody selected from the group consisting of mab 32, mab 22, mab 44, mab 62, mab 197, and anti-FcRI antibody 62.

18. The bispecific molecule of claim 17 wherein the monclonal antibody is mab 22.

19. The bispecific molecule of claim 15 wherein at least a portion of the all the complementarity determining regions of the human antibody are derived from a non-human antibody.

20. The bispecific molecule of claim 19 wherein the entire portion of all of the complementarity determining regions of the human antibody are derived from a non-human antibody.

21. The bispecific molecule of claim 19 wherein the non-human antibody is mab 22.

22. The bispecific molecule of claim 20 wherein the non-human antibody is mab 22.

23. The bispecific molecule of claim 12 wherein the target epitope is that of a cancer cell.

24. The bispecific molecule of claim 12 wherein the target epitope is that of an infectious agent.

25. The bispecific molecule of claim 12 wherein the target epitope is that of an antibody-producing cell.

26. The bispecific molecule of claim 12, wherein the target epitope is on a breast or ovarian cancer cell.

27. The bispecific molecule of claim 12, wherein the target epitope is HER 2/neu.

28. The bispecific molecule of claim 12, wherein the antigen binding region specific for a target epitope is antibody 520C9.
